(19) United States
(12) Patent Application Publication
(10) Pub. No.: US 2023/0124002 A1
(43) Pub. Date: Apr. 20, 2023

(54) CONTINUOUS-FLOW SYNTHESIS METHOD OF 13C-UREA

(71) Applicants: Shenzhen Zhonghe Headway Bio-Sci & Tech Co.,Ltd., Shenzhen (CN); Harbin Institute of Technology, Shenzhen, Shenzhen (CN)

(72) Inventors: Jing QING, Shenzhen (CN); Hengzhi YOU, Shenzhen (CN); Kai CHEN, Shenzhen (CN); Quansheng HAN, Shenzhen (CN); Guowei LI, Shenzhen (CN); Jingyuan LIAO, Shenzhen (CN); Xuelei JIA, Shenzhen (CN); Desheng DAI, Shenzhen (CN)

(21) Appl. No.: 18/067,293

(22) Filed: Dec. 16, 2022

(30) Foreign Application Priority Data
Jul. 26, 2022 (CN) 202210881735.4

(51) Int. Cl. C07C 273/02

(52) U.S. Cl. CPC C07C 273/02

(57) ABSTRACT

A continuous-flow synthesis method of ¹³C-urea, including: (S1) mixing sulphur and a methanol solution containing NH₃ in a feed kettle to obtain a slurry; or mixing ammonia gas, sulphur and methanol in a feed kettle to obtain a slurry; (S2) feeding the slurry into a mixing unit; and feeding ¹³CO into the mixing unit to obtain a three-phase mixture; (S3) mixing the three-phase mixture in the mixing unit evenly; feeding the three-phase mixture into a continuous-flow reactor for reaction to obtain a reaction product; and (S4) feeding the reaction product into a gas-liquid separator for gas-liquid separation, and collecting a liquid phase as a crude product solution; and subjecting the liquid phase to purification to obtain the ¹³C-urea.

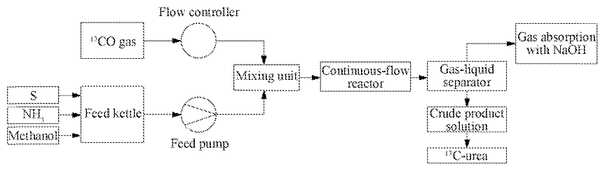

### CROSS-REFERENCE TO RELATED APPLICATIONS

This application claims the benefit of priority from Chinese Patent Application No. 202210881735.4, filed on Jul. 26, 2022. The content of the aforementioned application, including any intervening amendments thereto, is incorporated herein by reference in its entirety.

### TECHNICAL FIELD

This application relates to urea synthesis, and more particularly to a continuous-flow synthesis method of ¹³C-urea.

### BACKGROUND

Helicobacter pylori (H. pylori), as a spiral-shaped bacterium with numerous unipolar flagella, was discovered in 1982 by Barry J. Marshall and J. Robin Warren. H. pylori has been proved to be associated with gastritis, chronic gastroenteritis, gastric ulcer, duodenal ulcer, non-ulcer dyspepsia and some gastric cancers, and thus has attracted a lot of medical attention. Extensive researches have been conducted to investigate the relationship between H. pylori infection and gastrointestinal diseases over the past 30 years.

The H. pylori is detected and identified mainly by rapid urease test, H. pylori antibody test, ¹³C-urea (or ¹⁴C-urea) breath test, pathological tissue section method, and culture, where the ¹³C-urea (or ¹⁴C-urea) breath test high the highest precision (95-96% or more). ¹³C-urea is an essential raw material for the production of the ¹³C-urea breath test kit, but it is still difficult to achieve the industrial preparation of ¹³C-urea.

Generally, the urea is synthesized through the high-temperature and high-pressure reaction between carbon dioxide (CO₂) and ammonia (NH₃). Whereas, this synthesis route is not suitable for the ¹³C-urea preparation due to the poor CO₂ conversion.

### SUMMARY

In view of the defects in the prior art, the present disclosure provides a highly-efficient and safe continuous-flow synthesis method of ¹³C-urea. In this continuous-flow synthesis method route, ¹³CO, sulphur (S) and NH₃ are reacted in methanol in a continuous-flow reactor under heating and pressurizing conditions to continuously prepare ¹³C-urea, with 95-100% ¹³CO conversion rate, 90-95% ¹³C-urea yield and 99% ¹³C-urea purity. Therefore, the synthesis method provided herein has excellent product quality and high yield.

Technical solutions of the present disclosure are described as follows.

This application provides a continuous-flow synthesis method of ¹³C-urea, comprising:

(S1) mixing sulphur and a methanol solution containing NH₃ in a feed kettle to obtain a slurry; or mixing ammonia gas (NH₃), sulphur and methanol in a feed kettle to obtain a slurry;

(S2) feeding the slurry into a mixing unit; and feeding ¹³CO into the mixing unit to obtain a three-phase mixture;

(S3) mixing the three-phase mixture in the mixing unit evenly; and feeding the three-phase mixture into a continuous-flow reactor for reaction to obtain a reaction product; and

(S4) feeding the reaction product into a gas-liquid separator for gas-liquid separation, and collecting a liquid phase; subjecting the liquid phase to purification to obtain the ¹³C-urea.

In some embodiments, a molar ratio of the sulphur to NH₃ to ¹³CO is (1-5):(2-50):1.

In some embodiments, the continuous-flow reactor is controlled to 0-5 MPa and 50-150° C.

In some embodiments, in step (S2), a flow rate of the slurry is 0.001-10 L/min; and a flow rate of the ¹³CO is 0.001-100 L/min.

In some embodiments, a residence time of the three-phase mixture in the continuous-flow reactor is 1-120 min.

In some embodiments, in step (S4), the purification is performed through steps of:

subjecting the liquid phase to rotary evaporation in a rotary evaporator, dissolving with an alcohol or water, and vacuum filtration to collect a filtrate; and

drying the filtrate to obtain the ¹³C-urea.

In some embodiments, a purity of the ¹³C-urea after purification is 99%.

In some embodiments, a ¹³CO conversion rate is 95-100%; and a ¹³C-urea yield is 90-95%.

In some embodiments, a heat exchange medium of the continuous-flow reactor is heat-conducting oil or a water-based heat-conducting medium; and the water-based heat-conducting medium is water, a 1,2-ethanediol binary aqueous solution or a 1,2-propanediol binary aqueous solution.

In some embodiments, the slurry is fed by a feed pump into the mixing unit; the ¹³CO passes through a pressure reducing valve to enter the mixing unit, wherein a flow of the ¹³CO is controlled by a flow controller; and the reaction product flowing out from the continuous-flow reactor enters the gas-liquid separator through a back pressure valve.

Compared to the prior art, this application has the following beneficial effects.

1. This application additionally provides the mixing unit before the continuous-flow reactor to allow even mixing of the reaction materials, which can prevent the pipeline blockage caused by sedimentation, realizing the continuous ¹³C-urea synthesis. The continuous-flow synthesis method strategy provided herein has novel process, simple operation, high product quality and desirable yield, reducing the cost. Moreover, this application has simple purification, relatively mild reaction conditions, and low pollution. Therefore, the continuous-flow synthesis method provided herein is suitable for the industrial production of ¹³C-urea.

2. Compared to the batch reactor, the continuous-flow reactor enables the continuous-flow synthesis method, as well as the accurate control of the residence time of the reactants, allowing for improved yield and safety.

3. Compared to the batch reactor, the continuous-flow reactor can bring a higher ¹³C-urea yield, and can realize the continuous production of ¹³C-urea.

4. By means of the continuous-flow reactor, the reaction time is shortened from several hours to a few minutes, significantly enhancing the reaction efficiency and facilitating the large-scale production of ¹³C-urea.

5. The continuous-flow synthesis method provided herein enhances the mass transfer and heat transfer, and can keep the reaction temperature constant, thereby avoiding temperature runaway, material ejection and the loss of control, and effectively avoiding the leakage of toxic gases such as ¹³CO and H₂S. Moreover, the continuous-flow reactor has a small internal volume, such that the toxic substances exist in a relatively low level, greatly improving the operability and safety.

### BRIEF DESCRIPTION OF THE DRAWINGS

The drawings needed in the description of the embodiments of the disclosure or the prior art will be briefly described below to explain technical solutions of the embodiments of the present disclosure or the prior art more clearly. Obviously, presented in the accompany drawings are merely some embodiments of the present disclosure, and other drawings can be obtained by those skilled in the art based on the drawings provided herein without paying creative effort.

This FIGURE is a flow chart of a continuous-flow synthesis method of ¹³C-urea according to an embodiment of the present disclosure.

Implementation, features and advantages will be further illustrated below with reference to the accompany drawing and embodiments.

### DETAILED DESCRIPTION OF EMBODIMENTS

Technical solutions of the present disclosure will be clearly and completely described below with reference to the embodiments and accompanying drawings. Obviously, described below are merely some embodiments of this disclosure, and are not intended to limit the disclosure. Other embodiments obtained by those skilled in the art based on the embodiments provided herein without paying any creative effort should fall within the scope of the present disclosure.

Unless otherwise specified, the materials and reagents in the following embodiments are available commercially, and the experiments are performed by conventional methods. For the quantitative analysis, three replicates are performed, and the results are expressed as mean or mean±standard deviation.

As used herein, the “and/or” includes three solutions, for example, “A and/or B” includes A, B and a combination thereof. Additionally, technical solutions of various embodiments can be combined on the premise that the combined technical solution can be implemented by those skilled in the art. When the combination of technical solutions is contradictory or cannot be implemented, such a combination does not exist, and does not fall within the scope of the present disclosure.

Described herein was a continuous-flow synthesis method of ¹³C-urea, including steps of:

(S1) mixing sulphur and a methanol solution containing NH₃ in a feed kettle to obtain a slurry; or mixing ammonia gas (NH₃), sulphur and methanol in a feed kettle to obtain a slurry;

(S2) feeding the slurry into a mixing unit; and feeding ¹³CO into the mixing unit to obtain a three-phase mixture;

(S3) mixing the three-phase mixture in the mixing unit evenly; and feeding the three-phase mixture into a continuous-flow reactor for reaction to obtain a reaction product; and

(S4) feeding the reaction product into a gas-liquid separator for gas-liquid separation, and collecting a liquid phase; subjecting the liquid phase to purification to obtain the ¹³C-urea.

By replacing a kettle reactor with the continuous-flow reactor, a reaction time of continuous-flow synthesis is reduced from hours to minutes, significantly developing the reaction rate. In addition, the raw materials react in the continuous-flow reactor, contributing to a full contact therebetween. Particularly, the continuous-flow synthesis method using the continuous-flow reactor does not require gas compression into the reactor as opposed to the traditional kettle reactor reaction, shorting a process time, and improving a reaction efficiency.

Since a gas-phase raw material in the continuous-flow synthesis method is ¹³CO, the pressure in the continuous-flow reactor keeps constant during reaction and will not affect the reaction. To the contrary, regarding the reaction in the kettle reactor, ¹³CO therein will be consumed constantly, therefore, it is difficult to maintain the initial pressure in the reactor at the later stage of the reaction, resulting in reaction rate decreases and affecting the reaction efficiency.

In an embodiment, a molar ratio of the sulphur to NH₃ to ¹³CO is (1-5):(2-50):1.

In an embodiment, the continuous-flow reactor is controlled to 0-5 MPa and 50-150° C.

In an embodiment, in step (S2), a flow rate of the slurry is 0.001-10 L/min; and a flow rate of the ¹³CO is 0.001-100 L/min.

In an embodiment, a residence time of the three-phase mixture in the continuous-flow reactor is 1-120 min.

By using the above-mentioned technology solutions, the reaction time will be greatly reduced compared with traditional kettle-reactor reaction.

In an embodiment, in step (S4), the purification is performed through the following steps.

The liquid phase is subjected to rotary evaporation in a rotary evaporator, dissolving with an alcohol or water, and vacuum filtration to collect a filtrate. Then the filtrate is dried to obtain the ¹³C-urea.

In an embodiment, a purity of the ¹³C-urea after purification is 99%.

By using the above-mentioned technology solutions, the product yield is high.

In an embodiment, a ¹³CO conversion rate is 95-100%; and a ¹³C-urea yield is 90-95%.

In an embodiment, a heat exchange medium of the continuous-flow reactor is heat-conducting oil or a water-based heat-conducting medium; and the water-based heat-conducting medium is water, a 1,2-ethanediol binary aqueous solution or a 1,2-propanediol binary aqueous solution.

In an embodiment, the slurry is fed by a feed pump into the mixing unit; the ¹³CO passes through a pressure reducing valve to enter the mixing unit, wherein a flow of the ¹³CO is controlled by a flow controller; and the reaction product flowing out from the continuous-flow reactor enters the gas-liquid separator through a back pressure valve.

By using the above-mentioned technology solutions, the flow of the slurry and that of ¹³CO can be precisely controlled, thereby precisely controlling a reaction process.

In an embodiment, the continuous-flow synthesis method further includes a step of gas leak detection. The ¹³C-urea is synthesized in an operating room, which is kept in negative pressure by using an evacuating device. The evacuating device is communicated with an evacuating pipe. The operating room and the evacuating pipe are respectively provided with an ammonia sensing probe, a carbon monoxide sensing probe, and a hydrogen sulfide sensing probe. When levels of ammonia and/or carbon monoxide and/or hydrogen sulfide exceed a preset value, it indicates a possible gas leak. Since ammonia, carbon monoxide and hydrogen sulfide are hazardous to the health of operators, the system will give an alarm or a corresponding valve is immediately closed. The operators need to monitor and repair the pipe before continuing with the urea synthesis process.

The above-mentioned technology solutions can protect the operator from being damaged ammonia, carbon monoxide and hydrogen sulfide.

In summary, by means of the continuous-flow reactor, the continuous-flow synthesis method provided herein has novelty, simple operation, high product quality and yield, thereby leading to low cost and less pollution. By means of the continuous-flow reactor, the cost is greatly reduced. This application has simple operation, relatively mild reaction conditions, and relatively low pollution. The continuous-flow synthesis method provided herein effectively overcomes the defects of existing reactions that cannot be produced on a larger scale, facilitating large-scale production and improving quality and yield of the ¹³C-urea.

### EXAMPLE 1

(S1) 71.4 g of sulphur, 1200 mL of a methanol solution containing 7 mol/L of NH₃ and 1000 mL of methanol were fed into a feed kettle, and then mixed to obtain a slurry.

(S2) The slurry was fed by a feed pump into a mixing unit, to which ¹³CO was fed after passing through a pressure reducing valve, so as to obtain a three-phase mixture, where a flow rate of the ¹³CO was controlled at 0.1 L/min by a flow controller, and a flow rate of the slurry was 40 mL/min.

(S3) The three-phase mixture was mixed evenly in the mixing unit, fed into a continuous-flow reactor, and reacted at 130° C. and 3 MPa for 20 min to obtain a reaction product.

(S4) The reaction product was cooled by a cooling coil in an ice-water bath, and then flowed out of the continuous-flow reactor as a brown liquid to enter a gas-liquid separator for separation. A liquid phase was collected as a crude product solution, and subjected to rotary evaporation in a rotary evaporator, dissolving with water and vacuum filtration to collect a filtrate. The filtrate was dried to obtain ¹³C-urea. The gas phase generated from the gas-liquid separation was discharged and absorbed with a NaOH solution.

Examples 2-5 were performed according to the steps of Example 1. The amounts of raw materials and the reaction parameters of Examples 2-5 were shown in Table 1.

TABLE 1
Amounts of raw materials and reaction
parameters of Examples 1-5

| | Exam- | Exam- | Exam- | Exam- | Exam- |
|---|---|---|---|---|---|
| | ple 1 | ple 2 | ple 3 | ple 4 | ple 5 |
| Sulphur/g | 71.4 | 7.4 | 357 | 1071 | 3570 |
| Volume of the NH₃- | 1200 | 120 | 4000 | 12000 | 40000 |
| containing methanol | | | | | |
| solution/mL | | | | | |
| Concentration of | 7 | 7 | 7 | 7 | 7 |
| NH₃ in the methanol | | | | | |
| solution/mol · L⁻¹ | | | | | |
| Volume of ethanol | 1000 | 100 | 21000 | 63000 | 210000 |
| solution/mL | | | | | |
| Flow rate of slurry/ | 40 | 4 | 200 | 600 | 2000 |
| mL · min⁻¹ | | | | | |
| Flow rate of ¹³CO/ | 0.1 | 0.01 | 1 | 3 | 15 |
| L · min⁻¹ | | | | | |
| Reaction | 130 | 110 | 100 | 120 | 110 |
| temperature/° C. | | | | | |
| Pressure/MPa | 3 | 1 | 1 | 1 | 1 |
| Residence time/min | 20 | 20 | 40 | 40 | 40 |

¹³C-urea yields and ¹³CO conversion rates of Examples 1-5 were shown in Table 2.

TABLE 2
¹³C-urea yields and ¹³CO conversion rates of Examples 1-5

| | Exam- | Exam- | Exam- | Exam- | Exam- |
|---|---|---|---|---|---|
| | ple 1 | ple 2 | ple 3 | ple 4 | ple 5 |
| ¹³C-urea yield/% | 92.3 | 90.1 | 94.1 | 91.7 | 90.8 |
| ¹³CO conversion rate/% | 100 | 98 | 100 | 98 | 96 |

Comparative Examples 1-2 were performed basically according to the steps of Example 1.

Regarding Comparative Example 1, a slurry obtained in step (S1) and ¹³CO were directly fed into the continuous-flow reactor for reaction.

Regarding Comparative Example 2, it was free from addition of 1000 mL of methanol in step (S1).

¹³C-urea yields and ¹³CO conversion rates of Comparative Examples 1-2 are shown in Table 3.

TABLE 3
¹³C-urea yields and ¹³CO conversion rates of
Comparative Examples 1-2

| | Comparative | Comparative |
|---|---|---|
| | Example 1 | Example 2 |
| ¹³C-urea yield/% | 79.6 | 89.4 |
| ¹³CO conversion rate/% | 87 | 92 |

Technical solutions of various embodiments can be combined on the premise that the combined technical solution can be implemented by those skilled in the art. When the combination of technical solutions is contradictory or cannot be implemented, such a combination does not exist, and does not fall within the scope of the present disclosure.

Described above are only some embodiments of the present disclosure, which are not intended to limit the disclosure. Any variations and modifications made by those of ordinary skilled in the art without departing from the spirit of the disclosure should fall within the scope of the disclosure defined by the appended claims.

What is claimed is:

1. A continuous-flow synthesis method of ¹³C-urea, comprising:
(S1) mixing sulphur and a methanol solution containing NH₃ in a feed kettle to obtain a slurry; or mixing ammonia gas (NH₃), sulphur and methanol in a feed kettle to obtain a slurry;
(S2) feeding the slurry into a mixing unit; and feeding ¹³CO into the mixing unit to obtain a three-phase mixture;
(S3) mixing the three-phase mixture in the mixing unit evenly; and feeding the three-phase mixture into a continuous-flow reactor for reaction to obtain a reaction product; and
(S4) feeding the reaction product into a gas-liquid separator for gas-liquid separation, and collecting a liquid phase; subjecting the liquid phase to purification to obtain the ¹³C-urea.

2. The continuous-flow synthesis method of claim 1, wherein a molar ratio of the sulphur to NH₃ to ¹³CO is (1-5):(2-50):1.

3. The continuous-flow synthesis method of claim 1, wherein the continuous-flow reactor is controlled to 0-5 MPa and 50-150° C.

4. The continuous-flow synthesis method of claim 1, wherein in step (S2), a flow rate of the slurry is 0.001-10 L/min; and a flow rate of the ¹³CO is 0.001-100 L/min.

5. The continuous-flow synthesis method of claim 1, wherein a residence time of the three-phase mixture in the continuous-flow reactor is 1-120 min.

6. The continuous-flow synthesis method of claim 1, wherein in step (S4), the purification is performed through steps of:
subjecting the liquid phase to rotary evaporation in a rotary evaporator, dissolving with an alcohol or water, and vacuum filtration to collect a filtrate; and
drying the filtrate to obtain the ¹³C-urea.

7. The continuous-flow synthesis method of claim 6, wherein a purity of the ¹³C-urea after purification is 99%.

8. The continuous-flow synthesis method of claim 1, wherein a ¹³CO conversion rate is 95-100%; and a ¹³C-urea yield is 90-95%.

9. The continuous-flow synthesis method of claim 1, wherein a heat exchange medium of the continuous-flow reactor is heat-conducting oil or a water-based heat-conducting medium; and the water-based heat-conducting medium is water, a 1,2-ethanediol binary aqueous solution or a 1,2-propanediol binary aqueous solution.

10. The continuous-flow synthesis method of claim 1, wherein the slurry is fed by a feed pump into the mixing unit; the ¹³CO passes through a pressure reducing valve to enter the mixing unit, wherein a flow of the ¹³CO is controlled by a flow controller; and the reaction product flowing out from the continuous-flow reactor enters the gas-liquid separator through a back pressure valve.
